(19) United States
(12) Patent Application Publication
(10) Pub. No.: US 2019/0008655 A1
(43) Pub. Date: Jan. 10, 2019

(54) Spinal Cage Having Directed Apertures

(71) Applicant: Ossis Limited, Christchurch (NZ)

(72) Inventor: David Alistair John Body, Auckland (NZ)

(21) Appl. No.: 15/993,072

(22) Filed: May 30, 2018

(30) Foreign Application Priority Data
May 30, 2017 (NZ) 732359

(51) Int. Cl. A61F 2/44

(52) U.S. Cl. CPC A61F 2/4465; A61B 17/70; A61F 2/442

(57) ABSTRACT

A spinal implant for insertion between adjacent upper and lower vertebral endplates, the device including a first upper surface and a second opposing lower surface for contacting the upper and lower vertebral endplates respectively. The first and second opposing surfaces are connected by at least one external side wall. The at least one external side wall includes at least one entry aperture fluidly connected to at least one exit aperture on the upper and/or lower surfaces and/or internal or external side walls of the implant. The entry and exit apertures are fluidly connected by a conduit.

### CROSS-REFERENCE TO RELATED APPLICATIONS

This application incorporates by reference and claims the benefit of priority to NZ Patent Application No. 732359 filed on May 30, 2017.

### FIELD OF INVENTION

This invention relates to a spinal implant. More specifically the invention relates to a spinal implant having apertures to allow the transfer of material to exterior surfaces of the implant and methods for its use.

### BACKGROUND TO THE INVENTION

Spinal cages are used by orthopaedic surgeons to treat degenerative disc disease as well as spinal deformities and the impact of trauma. The purpose of the cage is to provide a load bearing structure, as well as providing a method to fuse adjacent vertebrae where the joint is unstable.

A recognised problem associated with spinal fusion is that the endplates of adjacent vertebrae the cage interfaces with can have very irregular surfaces. This means a spinal cage may not interface uniformly with the bone, leaving voids. The presence of voids between the cage surface and adjacent bone has implications with respect to load bearing and developing fusion between the bone and cage. In some circumstances, the spine endplates may “point load” onto a small area of the cage which could overload that part of the endplate, potentially causing fractures of the endplate or wall of the vertebrae, or subsidence of the cage through the endplates of the vertebrae above and/or below the cage. Collapse of the cage could also negatively impact sagittal balance along the patient's spine causing lower back or leg pain. In other scenarios, the void may prevent fusion of the joint, resulting in the spine remaining unstable at that point, which may lead to the patient continuing to exhibit their original symptoms such as back or leg pain.

The current state of the art is to pack voids manufactured within the cages with bone graft (autograft, allograft, bone morphogenic proteins, synthetic compounds and the like) which are used to promote fusion. However, the cages with spaces to pack bone graft must be preloaded with graft prior to insertion of the cage between adjacent vertebrae. When inserted, any excess material is “swept off” by the front faces of the adjacent vertebrae as the cage enters the intervertebral space. This leaves the remaining graft unable to fill any voids of spaces in the endplates due to the sweeping of the implant surface on entry. Alternatively, if the material is packed over the cage surfaces in an attempt to fill voids or depressions in the endplate, the surgeon could be tempted to over-distract the joint in an attempt to get around sweeping the material off. Over-distraction can weaken the ligaments that provide overall stability of the spine causing other long term problems, as well as jeopardising the fit and fusion of the cage between the adjacent vertebrae.

In circumstances where fusion of the cage to the surrounding vertebrae has not occurred successfully, a revision procedure is often undertaken, which increases the risk of tissue and ligament damage, as well as being detrimental to the patient. It would be further advantageous to develop an implant and method whereby the cage may be more firmly secured or re-secured against the vertebral endplates without removing or partially removing the initial cage.

### Object of the Invention

It is an object of the invention to provide a spinal cage that facilitates the application of material to vertebral endplates.

Alternatively, it is an object to provide a method for filling voids between spinal implants and vertebral endplates.

Alternatively, it is an object of the invention to at least provide the public with a useful choice.

### SUMMARY OF THE INVENTION

According to a first aspect of the invention, there is provided a spinal implant for insertion between adjacent upper and lower vertebral endplates, the device including; a first upper surface and a second opposing lower surface for contacting the upper and lower vertebral endplates respectively, the first and second opposing surfaces connected by at least one external side wall and optionally by internal side walls; wherein the at least one external side wall includes at least one entry aperture fluidly connected to one or more exit apertures on the upper and/or lower surfaces and/or internal or external side walls of the implant, the entry and exit apertures fluidly connected by a conduit.

Preferably, the entry aperture is located on the anterior side wall of the spinal

In further preferred embodiments the upper and/or lower surfaces of the spinal implant are solid, solid, porous or a combination thereof, with the exception of the exit aperture.

In one embodiment of the invention, the spinal implant includes a single exit aperture on the upper face or the lower face of the spinal implant.

In alternative embodiments of the invention the implant includes a single entry aperture and multiple exit apertures, the entry and exit apertures linked by a branched conduit.

In further alternative embodiments, the implant includes two or more entry apertures on the implant side wall, each entry aperture connected to one or more exit apertures on the upper and/or lower faces of the implant by straight or branched conduits.

In further embodiments the implant include two separate conduits, each accessing only the upper or lower implant surfaces independently.

In still further preferred embodiments, the exit apertures may include or be formed by open pathways in the porous structure of an implant.

In one embodiment the entry aperture includes a connection means adapted to receive a material injection mechanism. Preferably the connection means is in the form of a threaded wall portion or threaded insert, a female half of a click fit connection, an interference fit, compression fit or other connection means that corresponds to and connects with an injection system to be used for delivering filler material to the entry aperture.

In further embodiments, the exit apertures or walls thereof may be shaped to direct the flow of material to specific locations on the upper or lower implant surfaces.

In preferred embodiments the exit apertures in the upper and/or lower faces are positioned to correspond to predetermined voids or depressions in the endplates of the opposing vertebrae.

More preferably, the implant is a patient-specific implant and the position of the exit apertures are pre-determined using information derived from patient anatomy scans.

Alternatively, the implant is a non-custom implant and the position of the exit aperture(s) is predetermined based on information from scans in a population database.

Preferably, the internal conduit walls are smooth to enable optimal material flow within the conduit. In other embodiments the internal conduit walls have a spiralled surface or protrusions, for example, embedded fins, to improve and/or direct material flow through the conduit.

In some embodiments the one or more conduits have a substantially constant width along the length of the conduit.

In other embodiments, the conduit varies in width along the length of the conduit. Optionally, the conduit is narrower near the entry aperture(s) and wider near the exit aperture(s).

In further embodiments, the conduit may have a substantially circular cross section along all or part of its length. In alternative embodiments, the conduit may have an oval or irregular shaped cross section along its length.

In alternative embodiments, the conduit is in the form of a screw hole and includes a helical thread for receiving a screw. In this embodiment, the conduit may include a removable sheath designed for removal following the addition of a filler material, ensuring the screw thread remains viable for successful screw placement.

According to a further aspect of the invention, there is provided a method for improving fusion between a spinal implant and vertebrae, the method including the steps of:
a) locating a spinal implant between the endplates of a first and second vertebrae, the spinal implant having a first upper surface and a second opposing lower surface for contacting the upper and lower vertebral endplates respectively, the first and second opposing surfaces connected by at least one external side wall and optionally by internal side walls; wherein the at least one external side wall includes an entry aperture fluidly connected to one or more exit apertures on the first upper surface and/or an exit aperture on the second lower surface and/or internal or external side walls;
b) forcing a filler material through an entry aperture on a side wall and out an exit aperture to contact the endplates of the first and or second vertebrae.

Preferably, the filler material is selected from or comprised of one or more of bone graft, bone substitutes, bone morphogenetic protein (BMP), bone or other cement, elastomeric materials, therapeutic materials, antibiotic materials, materials for drug delivery or combinations thereof.

Preferably, the method includes forcing filler material through the entry aperture to fill voids between an endplate and an implant upper or lower surface.

In further embodiments of the invention, the entry and exit apertures are fluidly connected by a conduit as described in further detail above with reference to the spinal implant.

In further embodiments the conduit may also act as a screw hole, and the method includes a further step following step b) of; c) inserting a screw through an entry aperture and out the exit aperture of the implant and into the opposing vertebral endplate.

In alternative embodiments, step c) includes inserting a screw through the entry aperture to seal the entry aperture.

In some embodiments of the method, the method may include the further step of flushing or cleaning the conduit(s) following application of the filler material.

According to a still further aspect of the invention there is provided a method for the manufacture of a patient-specific spinal implant device for use between adjacent vertebrae, the method including the steps of;
a) imaging a patient's vertebrae around an implant insertion position using a 3D scanning technique to produce a patient specific scan;
b) reviewing the endplate surfaces of vertebrae adjacent the implant insertion position to identify surface irregularities;
c) designing a spinal implant device for insertion between the adjacent vertebrae, the device including; a first upper surface and a second opposing lower surface for contacting the adjacent vertebral endplates, the first and second opposing surfaces connected by at least one external side wall and optionally by internal side walls; wherein the at least one external side wall includes at least one entry aperture fluidly connected to one or more exit apertures on the upper and/or lower surfaces and/or internal or external side walls of the implant, the entry and exit apertures fluidly connected by a conduit; wherein the position of the one or more exit apertures corresponds with one or more surface irregularities identified in step b); and
d) building the implant designed in step c) using additive manufacturing.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 shows a top view of one embodiment of the spinal implant of the present invention;

FIG. 2 shows a bottom view of the spinal implant of FIG. 1;

FIG. 3 shows a side view showing the anterior face of the spinal implant of FIGS. 1 and 2;

FIG. 4 shows a vertical cross section of the spinal implant of FIG. 1-3;

FIG. 5 shows a perspective view of the spinal implant of FIGS. 1-4;

FIG. 6 shows a flow chart outlining the method for using the spinal implant of the present invention; and

FIG. 7 shows a flow chart outlining a method for the production of a patient-specific spinal implant in one embodiment of the invention.

### DETAILED DESCRIPTION OF PREFERRED EMBODIMENTS OF THE INVENTION

The spinal implant described herein is designed to improve outcomes in spinal interbody fusion procedures. The implant of the present invention provides a means for filling voids between the upper and lower surfaces of a spinal implant and the vertebral endplates each surface abuts when inserted between two vertebrae in a spinal column. The spinal implant discussed herein allows for void filling post implantation of the implant, helping develop regular loading of the connecting bone and encouraging bone development and successful fusion of the joint to the implant.

Spinal interbody fusion procedures may be completed by using a number of different surgical approaches, for example posterior lumbar interbody fusion (PLIF), anterior lumbar interbody fusion (ALIF), transforaminal lumbar interbody fusion (TLIF) and extreme lateral interbody fusion (XLIF). The implant of the present invention may be designed specifically for use with any of the above approaches, simply by positioning the entry aperture on the side wall of the implant that will be visible to the surgeon once the implant is in position; for example for an PLIF procedure the entry apertures will be located on the posterior side wall of the implant in order for the surgeon to locate and access the entry port successfully.

For the purposes of this specification where necessary we will discuss the design of the implant with reference to use in an ALIF procedure, however this is not intended to be limiting. Where possible, the positioning of the entry aperture on the implant will be in the region of the implant most accessible to the surgeon once the implant is in position. This may vary depending on the approach, as described above, but also on a case by case basis when a custom implant is being developed and a particular location will suit the specific procedure the best.

In a broad description the implant of the invention includes an entry aperture position on a side wall of a spinal implant, the entry aperture linked via one or more conduits to an exit aperture located on the upper and/or lower surface of the spinal implant. Once the implant has been position between two vertebrae, a filler material is forced through the entry aperture, into the conduit and out onto an upper and/or lower surface of the implant. The filler material extends across the implant surface and fills any holes or voids present in the endplates of the adjacent vertebrae or between the endplate of the adjacent vertebrae and the face of the implant. The ability to introduce the filler material after the implant has been seated means spaces can be successfully filled without losing material from a pre-coated implant as the implant is wedged between the vertebrae, and further reduces the need for distraction of the joints to enable material to be forced into the tight spaces between the implant and vertebral endplates. Also described is a method for utilising the implant in a surgical procedure.

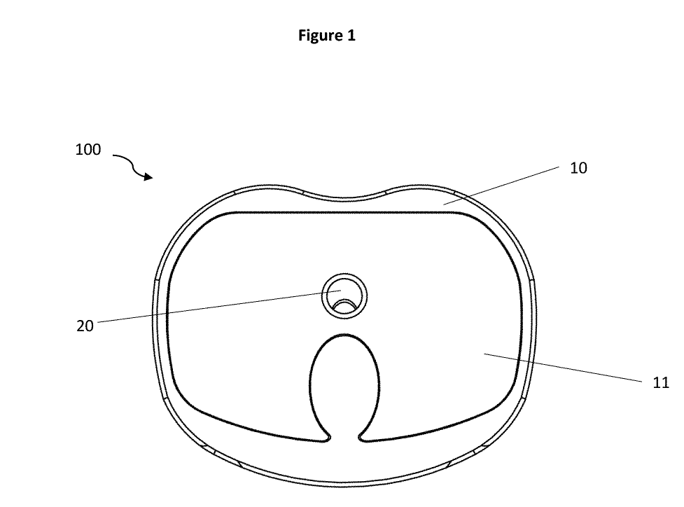

One embodiment of the invention can be seen in FIGS. 1-5 and is discussed in further detail below with reference to these figures. Implant 100, shown in different views across all figures, is a spinal implant formed using additive manufacturing techniques and is preferably formed from a combination of porous and solid metal, for example Ti6AlV4. Although additive manufacturing is the preferred form of the invention, the implant of the present invention may be formed using a range of materials currently used in spinal fusion implants, such as polyetheretherketone (PEEK), ceramics, polymers, metals and metal alloys such as chromium-cobalt alloys, stainless steel or titanium alloys, as well as a range of manufacturing techniques. Implants may be porous, solid or a combination thereof.

As range of different materials and techniques can be used to form the implant, the features of the invention may be used in both custom, patient-specific implants and off-the-shelf implants. Off-the-shelf implants may be formed with one or more exit apertures located in regions of the upper and lower endplates that are most commonly found across a significant portion of the population, and that are best located to provide optimum delivery of filler material to the endplates of a significant percentage of a specific population.

When used in the development of patient-specific implants, the positions and number of the exit apertures and therefore the path of the conduits within the implants can be optimised based on specific patient scan data. This may include utilising branched or straight conduits, multiple exit apertures on one face only, on both faces on the side wall(s) of the implant or may include variable size apertures at different locations of the implant surface for as a few non-limiting examples. As would be understood, and number or combinations or designs may be used to ensure the filler material is directed to the depressions and voids that would benefit from filling.

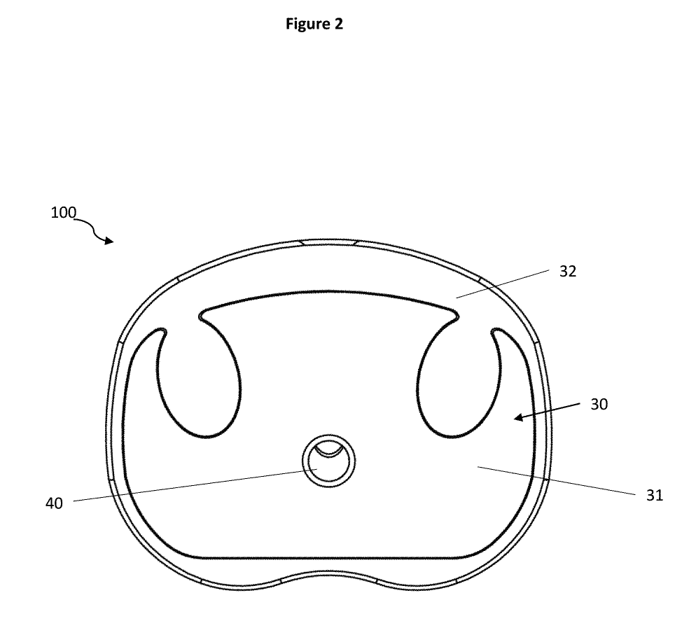

With reference to the figures provided, FIGS. 1 and 2 show a top and bottom view respectively of a titanium alloy ALIF spinal implant 100 formed by additive manufacturing with solid regions 10 and 32, and porous regions 11 and 31 on the upper and lower faces. Porous regions 11 and 31 on the upper and lower surfaces allow for bone growth between the vertebral endplates and the implant and for the filler material to integrate with both opposing surfaces. In other embodiments, the upper surface may be smooth, helping facilitate movement of the material into the voids and depressions between the surfaces and the endplates. The surfaces of the solid regions may be highly polished, rough textured, porous or combinations thereof and the surface used may be influenced by the filler material used. For example, filler materials of different viscosities may move more effectively into spaces when forced across surfaces with different finishes. When patient-specific implant design is utilised, the upper and lower surfaces may be formed with specific areas of texture and polish to aid in directing the filler material to the regions of the endplates that will result in optimum fixation. In some embodiments, grooves or channels may be formed on the upper and/or lower surfaces to further aid in directing filler material to a destination.

Upper surface 10 of implant 100 has a centrally located exit aperture 20, aperture 20 designed to provide an outlet for filler material (not shown) to exit and spread over surface 10 and into voids or depressions between the endplates and implant surface. FIG. 2 shows a bottom view of the same implant 100, with bottom surface 30 having a centrally located exit aperture 40 centrally located on the bottom surface 30.

The exit apertures may take a wide range of forms. The exit apertures may be formed as a simple spherical opening from the adjoining conduit, they may have sloped walls, a rounded lip or an upper rim that is shaped to specifically to encourage movement of filler material in specific directions. Likewise, the cross-sectional shape of the opening may also be specifically designed to provide optimal movement of the filler material. The exit apertures may range in size, for example, from 0.5 mm in diameter through to approximately 100 mm in diameter, with standard apertures likely to be in the range of 2 mm-6 mm in diameter. The upper and lower limits of the aperture sizes will be influenced and sometimes determined by the viscosity of the filler material, as well as the size restrictions imposed by the implant design itself. Larger aperture sizes are likely preferable, as this allows for the use of higher viscosity material, as well as decreasing the time taken for the filler material to be correctly distributed.

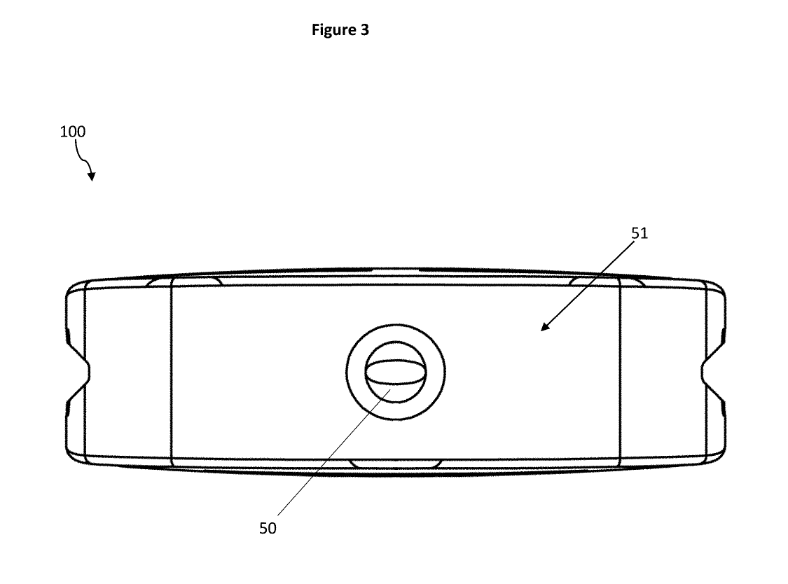

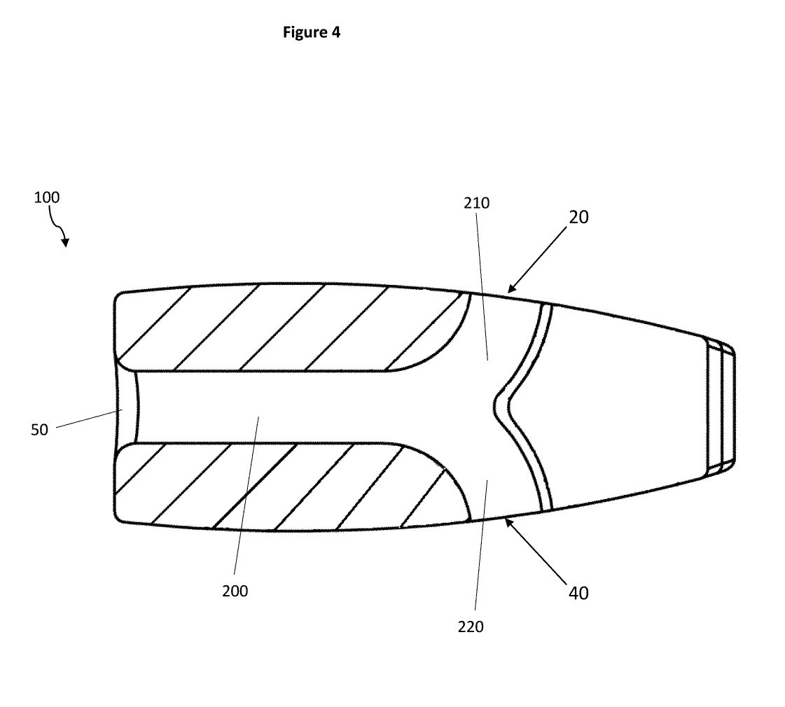

FIG. 3 shows an anterior side view of implant 100, with entry aperture 50 centrally located on the anterior face of side wall 51 of implant 100. Side wall 51 extends around the circumference of the upper surface 10 and lower surface 30. As shown by FIG. 4, entry aperture 50 is connected to exit apertures 20 and 40 on the upper and lower surfaces by conduit 200, which extends from entry aperture 50 through the implant body before branching into two conduits 210 and 220, which extend into exit apertures 20 and 40 respectively.

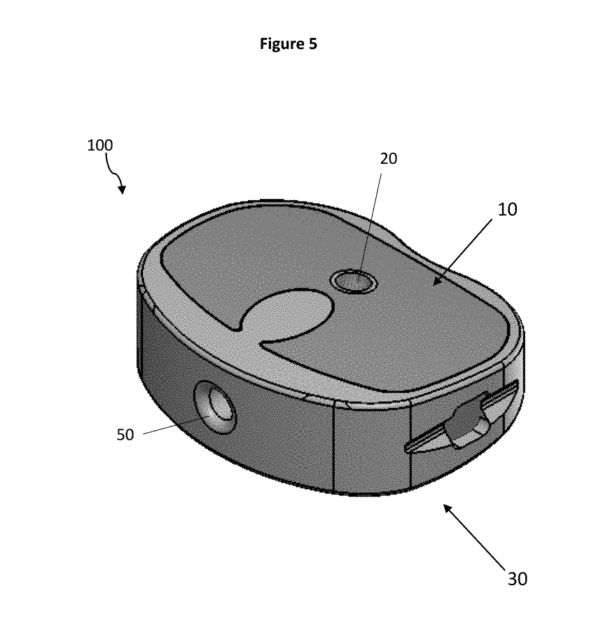

A perspective view of implant 100 is shown in FIG. 5 where entry aperture 50 is shown positioned on the anterior wall 51 allowing it to be easily accessed by the surgeon once positioned in the intervertebral space. In practice, entry aperture 50 may be formed on any face of the side wall that extends around the circumference of the upper and lower surfaces. The position of the entry aperture will be determined by the surgical approach taken for insertion of the implant (e.g. posterior or lateral), or in a patient-specific implant, may be positioned in a location that suits the requirements of a particular patient and/or patient case. Exit aperture 20 opens out onto upper surface 10, aperture 20 surrounded by a solid region of metal 12 on the upper surface 10.

As with the exit apertures above, the entry aperture(s) may be formed from a range of shapes and sizes and there may be more than one entry aperture depending on the size of the implant and the regions the filler material needs to be directed. Preferably, the implant includes a single aperture that will be effective in providing a single input to all exit apertures, decreasing the time required to perform the task of filling the voids.

The entry apertures may have an associated connection means that will allow connection with a corresponding tool for injecting the filler material. By providing a solid connection means, the filler material may be injected at a greater force without risk of the injection tool coming away from the implant during use, or the surgeon placing too much force on the implant once in position. Such connection means may be in the form of threaded wall portion or threaded insert, one half of a click fit connection or male-female connection, interference fit, compression fit or similar.

Conduits 210 and 220 may be formed with a smooth finish to enable optimal material flow within the conduit. In other embodiments the internal conduit walls have a spiralled surface or embedded fins to improve and/or direct material flow and/or mix through the conduit.

In some embodiments the one or more conduits have a substantially constant width along the length of the conduit, while in other embodiments, the conduit may vary in width along the length of the conduit. In one example, the conduit is narrower near the entry aperture(s) and wider near the exit aperture(s) to allow the material to reach greater surface areas once exiting the aperture.

In further embodiments, the conduit may have a substantially circular cross section along all or part of its length. In alternative embodiments, the conduit may have an oval or irregular shaped cross section along its length, particularly if changes in the conduit cross section are required to incorporate the desired size or number of conduits into the body of the implant.

In other examples envisaged, the exit apertures and/or conduits may include or be formed by open pathways in the porous structure of an implant. Such pathways may be singular or multiple and the porous structure is designed to ensure that filler material is directed to the preferred location on the implants upper and lower surfaces.

In some other examples of the invention, the entry aperture and/or conduit is in the form of a screw hole that has the ultimate purpose of receiving a screw for fixating the implant into the surrounding bone. Before insertion of the screws, the screw paths may be utilised as conduits for transporting filler material through the body of the implant. In such examples, the screw paths include a helical thread for receiving a screw. The conduit may also include a removable sheath designed for removal following the addition of a filler material, ensuring the screw thread remains viable for successful screw placement.

In alternative embodiments, the entry aperture and/or conduit is in the form of a screw hole designed to receive a screw for sealing the entry aperture to prevent filler material from regressing down the conduit and out the entry aperture, or to prevent entry of unwanted substances into the entry aperture.

Other sealing means such as plugs, caps, or quick drying filler material may also be added to the entry aperture and/or conduit to provide a seal.

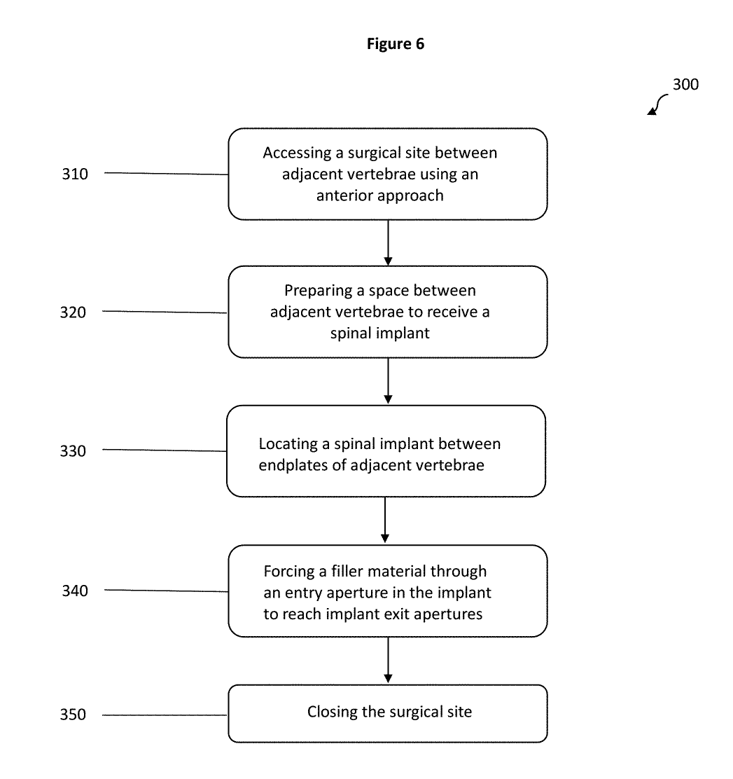

In use, for example in an ALIF procedure 300 as shown in FIG. 6, the surgical site is accessed using an anterior approach 310. Space for the spinal implant is prepared between two vertebral implants 320 by partially dissecting the annulus fibrosis and removing the inner nucleus pulposus. If necessary adjacent vertebrae are distracted and the spinal implant is inserted 330 between the endplates of two adjacent vertebrae, the anterior face of the implant 51 facing towards the surgeon once in position. Using a suitable injection means which may be directly connectable to the entry aperture, a filler material such as bone graft, bone cement, BMP, elastomeric materials or other therapeutic materials are injected through the entry aperture 340 with enough force to ensure the material is pushed through conduit 200 and exits the conduit through apertures 210 and 220. As upper and lower surfaces 10 and 30 are tightly positioned against the adjacent endplates, the filler material is forced into any voids and spaces between the two elements, significantly improving the connection between the two elements.

When the implant of the present invention is a custom implant, the fit between the implant and the endplates will be very accurate, and the filler material will be forced into the desired voids and depressions. For off the shelf implants, the fit will not always be as close. In these cases the filler material may fill out one side before the other. In such situations the material may be retained by the annulus fibrosis that is largely left in place by the surgeon. When this is not an option, excess filler material can be wiped away until the desired filling has occurred at all regions of the implant.

Once the filler material has been forced through the entry aperture and has reached the upper and lower endplates as required (judged by visual detection or once a certain pre-determined volume of filler material has been added), the injection means can be removed and the implant placement completed.

In alternative embodiments, wherein the exit apertures and/or conduits may include or be formed by open pathways in the porous structure of an implant, the filler material may be forced directly into the porous material of the implant using a tool, or it may be pushed in using the fingers.

The conduits of the implant may then optionally be flushed with saline or other solution to ensure the conduits and entry/exit ports remain open. This then allows for the option of reintroducing further filler material at a later point should the implant require further stabilisation. In cases where a conduit sheath is used before the filler material is injected, the sheaths can then be removed before the procedure is finished, again readying the conduits for further use if required. The surgical site is then closed 350.

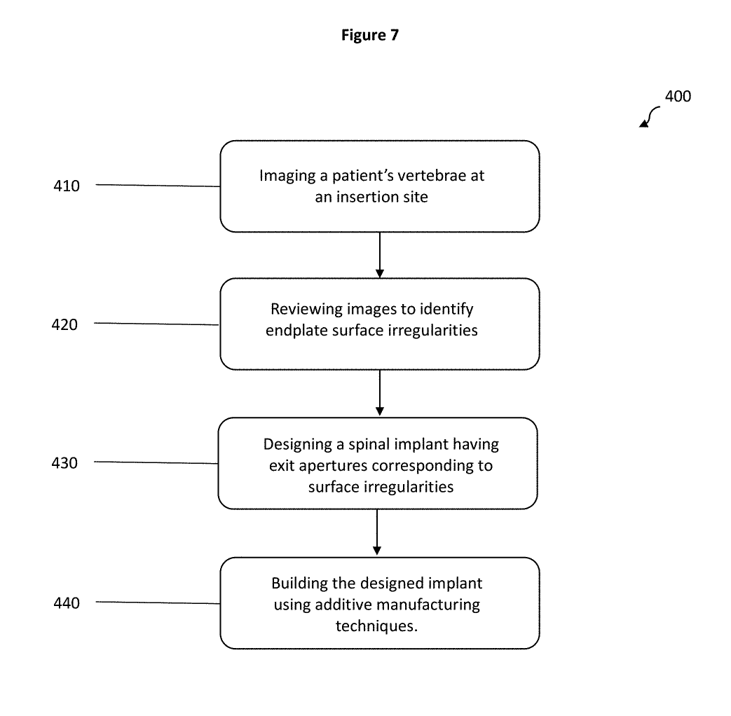

The method of manufacture 400 of a patient-specific 3D spinal implant of the present invention is outlined in FIG. 7. In creating a patient specific implant, a scan is taken of the spinal area where the implant is taking place 410. This may be X-ray, computed tomography (CT) or MRI scanning, either alone or in combination as required in order to gain an accurate representation of the patient's spine, particularly the endplate surfaces between which the spinal implant will be inserted.

The scanned images are reviewed to identify any irregularities in the areas of the vertebrae that will abut a spinal implant once positioned 420. These areas may be depressions or protrusions that may prevent successful contact between the implant and bone, resulting in less the optimal stabilisation of the joint due to bone ingrowth with the implant. In areas where depressions are found, exit apertures are positioned at or near the region of the depression, such that in use, the filler material is directed to an area that will benefit from the addition of additional filler material. For areas of protrusion, the implants are designed with exit apertures adjacent the protruding area, to enable filler material to fill any voids created by the protrusion in the surrounding areas.

The patient specific implant is then designed 430 with these features taken into account, with the position of the exit apertures, path of the conduits, or pathways through the porous material designed to direct filler material to patient specific regions. The position of the exit apertures may be the same or different on the upper and lower surfaces, there may be multiple apertures on a single face, or apertures on one face only as required by the vertebral endplates.

Once the implant design has been completed, a model is optionally made, then the implant itself is created using known additive manufacturing techniques such as EBM or direct metal laser sintering (DMLS). Preferably, the implants are formed from titanium Ti6Al4V, however the implant design as described herein could be implemented using a range of different materials.

Following manufacture of the augment, the augment is then surface finished if necessary and any further features added such as screw placement holes are machined in. The augment is then cleaned and sterilised before being provided to a hospital or surgical professional for use.

The spinal implant described herein has a number of advantages over the prior art and current state of the art. By providing a means to fill voids and increase and improve contact between an interbody fusion cage and the adjacent endplates, without over distracting the joints, risking subsidence or without having to preload an implant with a filler material, stronger and more successful interbody fusion can be achieved.

In addition to improving the performance of newly introduced implants, the implant system may also be used as an alternative when a patient would normally require a revision procedure. Bone graft may be reinjected into the conduit system in order to improve or add to bone growth between the implant and vertebrae, alternatively, bone cement may be added to secure a loosening implant for example. This significantly reduces the risk to the patient, as the implant can be secured further without the need for a full removal and replacement.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

I claim:

1. A spinal implant for insertion between adjacent upper and lower vertebral endplates, the device including;
a first upper surface and a second opposing lower surface for contacting the upper and lower vertebral endplates respectively, the first and second opposing surfaces connected by at least one external side wall;
wherein the at least one external side wall includes at least one entry aperture fluidly connected to at least one exit aperture on the upper and/or lower surfaces and/or internal or external side walls of the implant, the entry and exit apertures fluidly connected by a conduit.

2. The spinal implant of claim 1, wherein the entry aperture is located on the anterior side wall of the spinal implant.

3. The spinal implant of claim 1, wherein the implant includes a single exit aperture on the upper face or the lower face of the spinal implant.

4. The spinal implant of claim 1, wherein the implant includes a single entry aperture and multiple exit apertures, the entry and exit apertures linked by a branched conduit.

5. The spinal implant of claim 1, wherein the implant includes two or more entry apertures on the implant side wall, each entry aperture connected to one or more exit apertures on the upper and/or lower faces of the implant by straight or branched conduits.

6. The spinal implant of claim 1, wherein the implant includes two entry apertures and two exit apertures, a first entry aperture connected via a first conduit to a first exit aperture on the upper face of the implant and a second entry aperture connected via a second conduit to a second exit aperture on the lower face of the implant, the first and second conduits being independently from each other.

7. The spinal implant of claim 1, wherein the exit apertures may include or are formed by open pathways in the porous structure of an implant.

8. The spinal implant of claim 1, wherein the entry aperture includes a connection means adapted to receive a material injection mechanism.

9. The spinal implant of claim 1, wherein, the exit apertures or walls thereof are shaped to direct the flow of material to specific locations on the upper or lower implant surfaces.

10. The spinal implant of claim 1, wherein, the implant is a patient-specific implant and the position of the exit apertures are pre-determined using information derived from patient anatomy scans.

11. The spinal implant of claim 1, wherein the implant is a non-custom implant and the position of the exit aperture(s) is predetermined based on information from scans in a population database.

12. The spinal implant of claim 1, wherein the internal conduit walls are substantially smooth.

13. The spinal implant of claim 1, wherein the internal conduit walls have a spiralled surface or protrusions extending from the conduit walls.

14. The spinal implant of claim 1, wherein the conduit is in the form of a screw hole and includes a helical thread for receiving a screw.

15. The spinal implant of claim 1, wherein the conduit includes a removable sheath adapted for removal following the addition of a filler material.

16. A method for improving fusion between a spinal implant and vertebrae, the method including the steps of;
a) locating a spinal implant between the endplates of a first and second vertebrae, the spinal implant having a first upper surface and a second opposing lower surface for contacting the upper and lower vertebral endplates respectively, the first and second opposing surfaces connected by at least one external side wall and optionally by internal side walls; wherein the at least one external side wall includes an entry aperture fluidly connected to one or more exit apertures on the first upper surface and/or an exit aperture on the second lower surface and/or internal or external side walls;
b) forcing a filler material through an entry aperture on a side wall and out an exit aperture to contact the endplates of the first and or second vertebrae.

17. The method of claim 16, wherein the filler material is selected from or comprised of one or more of bone graft, bone substitutes, bone morphogenetic protein (BMP), bone or other cement, elastomeric materials, therapeutic materials, antibiotic materials or materials for drug delivery.

18. The method of claim 16, wherein the conduit is a screw hole, and the method includes a further step following step b) of; c) inserting a screw through an entry aperture and out the exit aperture of the implant and into the opposing vertebral endplate.

19. According to a still further aspect of the invention there is provided a method for the manufacture of a patient-specific spinal implant device for use between adjacent vertebrae, the method including the steps of;
a) imaging a patient's vertebrae around an implant insertion position using a 3D scanning technique to produce a patient specific scan;
b) reviewing the endplate surfaces of vertebrae adjacent the implant insertion position to identify surface irregularities;
c) designing a spinal implant device for insertion between the adjacent vertebrae, the device including; a first upper surface and a second opposing lower surface for contacting the adjacent vertebral endplates, the first and second opposing surfaces connected by at least one external side wall and optionally by internal side walls; wherein the at least one external side wall includes at least one entry aperture fluidly connected to one or more exit apertures on the upper and/or lower surfaces and/or internal or external side walls of the implant, the entry and exit apertures fluidly connected by a conduit; wherein the position of the one or more exit apertures corresponds with one or more surface irregularities identified in step b); and
d) building the implant designed in step c) using additive manufacturing.
